(19) United States
(12) Patent Application Publication
(10) Pub. No.: US 2021/0217489 A1
(43) Pub. Date: Jul. 15, 2021

(54) SAMPLE INDEXING METHODS AND COMPOSITIONS FOR SEQUENCING APPLICATIONS

(71) Applicant: 10X Genomics, Inc, Pleasanton, CA (US)

(72) Inventors: Michael Schnall-Levin, San Francisco, CA (US); Lawrence Greenfield, San Mateo, CA (US)

(21) Appl. No.: 17/002,641

(22) Filed: Aug. 25, 2020

Related U.S. Application Data
(63) Continuation of application No. 15/135,858, filed on Apr. 22, 2016, now abandoned.
(60) Provisional application No. 62/151,867, filed on Apr. 23, 2015.

(51) Int. Cl. G16B 30/00; C12Q 1/6874; C12N 15/10; C12Q 1/6806

(52) U.S. Cl. CPC G16B 30/00; C12Q 1/6806; C12N 15/1093; C12Q 1/6874

(57) ABSTRACT

Compositions, processes and systems are provided for preparing and analyzing sample indexing of nucleic acid libraries for multiplexed sequencing analysis of diverse sample sets.

### CROSS-REFERENCE TO RELATED APPLICATIONS

This application is a continuation of U.S. patent application Ser. No. 15/135,858, filed Apr. 22, 2016, which claims the benefit of U.S. Provisional Application No. 62/151,867, filed Apr. 23, 2015, both of which are hereby incorporated by reference in its entirety for all purposes.

### STATEMENT REGARDING FEDERALLY SPONSORED RESEARCH

Not Applicable.

### BACKGROUND OF THE INVENTION

Nucleic acid sequencing has made unprecedented advancements over the past decade, bringing high throughput, relatively low cost DNA sequence information to researchers, diagnosticians and health care professionals. Despite increased throughput of modern sequencing technology, there are always challenges in further multiplexing the analytical process, in order to be able to analyze more sequences and more samples.

By way of example, in current sequencers, shorter fragments of an overall sample nucleic acid, are sequenced and re-assembled to provide the sequence of the original sample nucleic acid. In order to sequence larger numbers of different samples, it is useful to pool samples in a single sequencing run. However, in order to do this without sequence information from different samples confounding the analysis of each other, the fragments from each different sample are provided with a unique oligonucleotide sequence appended to one end of the sequence, which identifies the sample of origin from the sequences obtained from the pooled samples. This unique sequence is read during the sequencing process, providing an index for a given read that attributes that read to a given starting sample.

While this sample indexing process has proven effective, a difficulty arises in some of the sequence data processing systems associated with available short read sequencing systems. In particular, these processing systems often fail when the sequence data includes multiple disparate sequences having identical nucleotides at a given position. In particular, where a significant percentage of the discrete sequences being read in a given sequencing run, e.g., at different oligonucleotide clusters within a given flow-cell, have identical nucleotides at the same sequence position, it can result in analytical failures of the base calling software for these systems. In particular, the systems are unable to process data where a significant number of the clusters share the same nucleotides at the same position, and as a result, render the bases at those positions un-callable. Given the complexity of the genome and the numbers of sequences typically analyzed in a given sequencing run, this failure mode is not routinely encountered in the analysis of sample sequences.

However, this limitation does put significant constraints on the use of any common sequence elements in significant portions of the disparate sequences being analyzed, such as primer sequences, index sequences and the like. By way of example, this limitation does significantly impact the selection of sample index sequences that one may use in performing multiplexed sample sequencing, by requiring that a given sample include multiple sample indices that are selected so that there are reduced overlapping sequence elements. This has the effect of providing limits on the sample multiplex level for a sequencing reaction.

Provided herein are solutions to these and other shortcomings of current sequencing processes.

### BRIEF SUMMARY OF THE INVENTION

Described herein are processes, compositions and systems for use in multiplexed sequence analysis of diverse sets of sample nucleic acids. In particular, provided herein are universal sample index sets and libraries that provide sequence diversity as between index sequences in a given set and as between different sets of index sequences, allowing a greater ability to multiplex sequence analysis.

In one aspect, the present disclosure provides a universal sample index library that includes a plurality of sets of sample index oligonucleotides, where each of the plurality of sets of sample index oligonucleotides includes a plurality of individual sample index oligonucleotide sequences. In some aspects, the sample index oligonucleotides in each of the plurality of sets of sample index oligonucleotides are different from sample index oligonucleotides in each other set of sample index oligonucleotides. In further aspects, each sample index oligonucleotide sequence within a set of sample index oligonucleotides includes a different nucleotide sequence from each other sample index oligonucleotide in the same set of sample index oligonucleotides.

In a further aspect, the present disclosure provides a method of sample indexing oligonucleotides for nucleic acid sequencing that includes the steps of (i) providing a plurality of sequencing libraries of oligonucleotides, each of the plurality of sequencing libraries being prepared from a different sample and (ii) attaching sets of sample index oligonucleotides to each of the plurality of sequencing libraries of oligonucleotides. In a further exemplary aspect, the sample index oligonucleotides in each of the plurality of sets of sample index oligonucleotides are different from sample index oligonucleotides in each other set of sample index oligonucleotides; and each sample index oligonucleotide sequence within a set of sample index oligonucleotides comprises a different nucleotide sequence from each other sample index oligonucleotide in the set of sample index oligonucleotides. In an exemplary embodiment, after the attaching step, the sequencing libraries of oligonucleotides are pooled together and subjected to a sequencing process.

In a further embodiment, and in accordance with any of the above, each set of sample index oligonucleotides includes at least three, four, five, six, seven, eight, nine, or ten different sample index oligonucleotides.

In a still further embodiment, and in accordance with any of the above, the plurality of sets of sample index oligonucleotides comprises at least about 10 sets, 20 sets, 50 sets, or 100 sets.

In a yet further embodiment, and in accordance with any of the above, each of the plurality of sets of sample index oligonucleotides has complete diversity from other sets of the plurality.

In a still further embodiment, and in accordance with any of the above, each sample index oligonucleotide within a set of sample index oligonucleotides comprises a different nucleotide at each sequence position from each other sample index oligonucleotide in the set of sample index oligonucleotides.

In a further embodiment, and in accordance with any of the above, each sample index oligonucleotide within a set of sample index oligonucleotides does not share a common 4-mer sequence with any other sample index oligonucleotide within that same set of sample index oligonucleotides.

In a still further embodiment, and in accordance with any of the above, the sample index oligonucleotides within a set have less than 80% common bases at common sequence positions with other sample index oligonucleotides within the same set.

In a yet further embodiment, and in accordance with any of the above, the sample index oligonucleotides are from about 4 to about 10 bases in length.

In a still further embodiment, and in accordance with any of the above, the sample index library further includes adapter sequences containing additional sequence elements. In a further exemplary embodiment, the sample index oligonucleotides are integrated into the adapter sequences.

### DETAILED DESCRIPTION OF THE INVENTION

### I. General

Provided herein are improved sample indexing compositions, methods and systems that alleviate the informatics problems associated with current indexing systems. As described above, the presence of excessive amounts of common sequences in certain next generation sequencer runs, can lead to a failure of the data processing systems, and particularly to the base calling software. This is particularly problematic where common sequences are introduced into significant portions of the sequences in a given sequencing run. Of particular note are sample index sequences where a common sample index is typically tagged with a single short, common, sequence tag of from about 4 to about 10 nucleotides in length, and typically from 6 to 8 nucleotides in length. Introduction of this common sequence across a large number of the sequence fragments being run in a given analysis run can lead to the failures described above.

As described herein, provided are sets of sample index oligonucleotides, where each set is used to index a library of oligonucleotides for sequencing from a given individual sample. Within each set are a plurality of different sample index oligonucleotides that differ from each other at every nucleotide within their sequence, or a significant portion of the nucleotides within the sequence. For example, assuming a first sample index set having a first 8-mer having the sequence:

| | |
|---|---|
| INDEX 1: | GAACGTAC |

The set may also include one or more of sample index sequences that vary at one or more positions. For example, as shown below, a set is illustrated which varies at each and every position:

| | | | | | | | | |
|---|---|---|---|---|---|---|---|---|
| INDEX 1 | G | A | A | C | G | T | A | C |
| INDEX 2 | A | T | T | G | A | C | T | G |
| INDEX 3 | T | C | C | A | T | G | C | A |
| INDEX 4 | C | G | G | T | C | A | G | T |

Although illustrated as an 8-mer, it will be appreciated that the sample index sequences will typically be from about 4 to about 10 bases in length, and preferably are from about 6 to about 8 bases in length, inclusive, though such index sequences can be varied in length outside of these ranges as desired, depending upon the number of different samples that are desired to be analyzed simultaneously, and the sequence read-length requirements of the given analysis. In particular, using a short read sequencing technology, longer index sequences may reduce the length of the sequence reads that may apply to the sample sequence portion of the analysis.

Although illustrated above as 4 discrete sample index sequences in a set, a given set of sample index sequences may include fewer than 4 sequences or may include additional index sequences that vary at each position or a sufficient number of positions. In certain cases, it will be desired that as between index sequences in a given set, e.g., applied to a single sample, there will be a common base at a common position no more than 80% of the time (e.g., with respect to a given sequence position in a set of index sequences, 80% or less of those positions may include a common base). In many cases, as between index sequences in a given set, there will be a common base at a common position no more than 70% of the time, no more than 60% of the time, no more than 50% of the time, no more than 40% of the time, no more than 30% of the time, no more than 20% of the time, no more than 10% of the time. In still further cases, in some sample index sets, as between different sequences in that set, no sequence positions will share a common base. By way of example, for an 8-mer sample index, as between sample indices in a given set of sample index sequences, the different indexes in the set may have overlap, or common bases at the same position at 6 bases or fewer, at 5 bases or fewer, at 4 bases or fewer, at 3 bases or fewer, at 2 bases or fewer, at 1 base or fewer, and in certain cases, will vary at each and every base. Rephrased, with respect to index sequences of from about 6 to about 10 bases in length, this may result in sequences that do not have common bases in 2, 3, 4, 5, 6, and as the case may be, 7, 8, 9 or 10 common sequence locations within the index sequences in a set.

In still further cases, the index sequences in a given set will not share a common 4-mer sequence, i.e., in the same positions, will not share a common 3-mer sequence, or will not share a common 2-mer sequence of bases within the index sequences, while in other cases, such common n-mer sequences will be present in fewer than 20% of the index sequences in the set, fewer than 10% of the index sequences in the set or fewer than 5% of the index sequences in the set. By “n-mer” as used herein is meant a series of “n” contiguous bases within the index sequence.

As between different sets of sample indices being applied to a given sequencing run, e.g., applied to different samples run on a single flow cell, the sequences will also vary such that all index sequences in a first set will be different from all index sequences in a second set. The level of difference between sets will typically provide sample indices at different clusters that have common nucleotides at common positions less than 80% of the time, preferably, less than 70% of the time, less than 60% of the time, less than 50% of the time, less than 40% of the time, less than 30% of the time, less than 20% of the time, less than 10% of the time, and in some cases, will differ at each and every base in the index sequences present in the different sets. By way of example, for an 8-mer sample index, as between sample indices in a given sequencing run, the different sets of sample indices present in a sequencing run would typically have overlap, or common bases at the same position at 6 bases or fewer, at 5 bases or fewer, at 4 bases or fewer, at 3 bases or fewer, at 2 bases or fewer, at 1 base or fewer, and in certain cases, will vary at each and every base. Rephrased, with respect to index sequences of from about 6 to about 10 bases in length, this may result in sequences that do not have common bases in 2, 3, 4, 5, 6, and as the case may be, 7, 8, 9 or 10 common sequence locations as between the index sequences in different sets.

By virtue of providing sequence variability within a given set of sample index sequences used for a given sample, one alleviates the need to mix and match sample index sequences to reduce data analysis problems. In particular, a ready made, universal set of diverse sample index sequences is provided for use with each given sample, with diversity that is tailored for the analysis, including, e.g., complete diversity, i.e., variation at each base of the sample index sequences.

As noted above, a given sample index set will preferably have 2, 3, 4 or more diverse index sequences included therein. Likewise, a given set or group of sets may be selected from a library of sets that may vary depending upon the given analysis, and as described above. Generally, the number of sets in the library of sets of sample index sequences will typically include at least about 10, 20, 30, 40, 50, 60, 70, 80, 90, 100, 200, 300, 400, 500, 750, 1000, 1500, 2000, 2500, 3000 or more different sets of sample index sequences, and in many cases will be between the above described numbers of sets and up to 10,000 different sets or even more.

In use, a given sample index set may be used in identifying a single discrete nucleic acid sample, e.g., from a single patient, a single tissue sample, a single cell, or the like. Different samples would be identified using a discrete set of sample index sequences. Upon sequencing of a pooled set of samples, attribution of the sequence information obtained to the originating sample would be carried out by identifying the set from which the index sequence belongs. As such, rather than identifying a single index sequence as being attributed to a given starting sample, e.g., patient, tissue sample, cell, etc., one would identify a given set of unique sample index sequences as being attributable to a given starting sample.

The sample index sequences described herein are typically provided within the context of larger adapter sequences that include additional sequence elements that permit the appending of the adapter sequence to sequencing library elements, and that provide additional sequence elements necessary for the sequencing process, e.g., flow cell attachment sequences, sequencing primer sequences, and the like. In such cases, the index sequence will typically be positioned at a sequenced location, e.g., located downstream, or 5′, of the relevant sequencing primer sequence for a given sequence read, so that the index sequence will be included with the overall sequence data.

For example, the sample index sets described herein may be readily integrated into the adapter sequences used in a conventional sequencing library workflow. Briefly, these workflows typically provide fragments of nucleic acids from a given sample. These fragments are processed to append appropriate sequence segments on one or both ends of the sample nucleic acid fragments. Typically, these sequence segments can include the sequencer functional elements, such as attachment sequences and sequencing primer recognition sequences (also referred to herein as primer sequences). Sample index sequences are also typically appended to one or both ends of the nucleic acid fragments from a given sample. Upon sequencing, the sequence of the sample nucleic acid fragment is determined along with the sequence of the appended sample index sequence, which allows attribution of the sample nucleic acid sequence data back to the particular sample. By appending different index sequences to different samples, it allows pooling of multiple discrete samples onto a single sequencing run, while allowing attribution of the resulting sample sequence information to a given sample. As described herein, different sets of sample index sequences would be appended to the nucleic acids from each sample.

By way of example, these sample index sets may be integrated into the adapter sets used in the Illumina TruSeq® DNA Sample Preparation kits used in the Illumina sequencing processes, where dual index adapters are ligated to opposing ends of double stranded sample nucleic acid fragments. Likewise, these sample index sequence sets may be integrated into other adapter sequences used in any other sample index workflow step for other sequencing library preparation processes where a greater diversity of the index sequences is desired. In an additional example, those sequence library preparation processes described in, e.g., U.S. patent application Ser. No. 14/316,383, filed Jun. 26, 2014, Ser. No. 14/752,589, filed Jun. 26, 2015, and U.S. patent application Ser. No. 14/990,276, filed Jan. 7, 2016, the full disclosures of which are incorporated herein by reference in their entirety for all purposes, may employ the index sequence sets described herein in the adapter sequences appended to barcoded sequence libraries along with the additional sequence components appended to those library elements, e.g., attachment sequences and sequencing primer sequences.

Thus, in some cases, provided herein are sample index sequence compositions that include sets of oligonucleotides that include a sample index sequence where each oligonucleotide in the set differs from each other oligonucleotide in the set within at least the sample index sequence portion. In particular, each sample index sequence within a set will differ from each other sample index within the set at every nucleotide within their sequence or a significant portion of the nucleotides within the sequence as described elsewhere herein, and preferably will vary at each and every base within the sample index sequence.

As noted previously the sets of oligonucleotides may comprise adapter sequences that include additional functional sequences as described above, where the index portions are oriented within the oligonucleotides such that they will be subjected to sequence determination in a sequencing process, e.g., downstream of a sequencing primer sequence for a given sequence read.

The compositions described herein may be provided in a kitted format as a portion of sequence library preparation kits or systems, or as kits for sample indexing in their own right. Such kits may include the compositions described herein as sample index sequences, as adapter sequences, or the like, so that they may be integrated into workflows for use in analysis, e.g., in sequencing protocols. The kits described herein may also include additional reagents used in the library preparation process, e.g., as provided in TruSeq sample preparation kits available from Illumina, Inc., or in sequence library preparation systems, e.g., as described in U.S. patent application Ser. No. 14/316,398, filed Jun. 26, 2014, the full disclosure of which is incorporated herein by reference in its entirety for all purposes.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. For example, any of the sample index sequences described herein can be used in conjunction with any sequencing platforms described herein and known in the art. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

What is claimed is:

1. A universal sample index library, comprising a plurality of sets of sample index oligonucleotides, each of the plurality of sets of sample index oligonucleotides comprises a plurality of individual sample index oligonucleotide sequences, wherein:
the sample index oligonucleotides in each of the plurality of sets of sample index oligonucleotides are different from sample index oligonucleotides in each other set of sample index oligonucleotides; and
each sample index oligonucleotide sequence within a set of sample index oligonucleotides comprises a different nucleotide sequence from each other sample index oligonucleotide in the same set of sample index oligonucleotides.

2. The library of claim 1, wherein each set of sample index oligonucleotides comprises at least three, four, five, six, seven, eight, nine, or ten different sample index oligonucleotides.

3. The library of claim 1, wherein the plurality of sets of sample index oligonucleotides comprises at least about 10 sets, 20 sets, 50 sets, or 100 sets.

4. The library of claim 3, wherein each of the plurality of sets of sample index oligonucleotides has complete diversity from other sets of the plurality.

5. The library of claim 1, wherein each sample index oligonucleotide within a set of sample index oligonucleotides comprises a different nucleotide at each sequence position from each other sample index oligonucleotide in the set of sample index oligonucleotides.

6. The library of claim 1, wherein each sample index oligonucleotide within a set of sample index oligonucleotides does not share a common 4-mer sequence with any other sample index oligonucleotide within that same set of sample index oligonucleotides.

7. The library of claim 1, wherein sample index oligonucleotides within a set have less than 80% common bases at common sequence positions with other sample index oligonucleotides within the same set.

8. The library of claim 1, wherein the sample index oligonucleotides are from about 4 to about 10 bases in length.

9. The library of claim 1, wherein the sample index library further comprises adapter sequences containing additional sequence elements.

10. The library of claim 8, wherein the sample index oligonucleotides are integrated into the adapter sequences.

11. A method of sample indexing oligonucleotides for nucleic acid sequencing, comprising:
providing a plurality of sequencing libraries of oligonucleotides, each of the plurality of sequencing libraries being prepared from a different sample;
attaching sets of sample index oligonucleotides to each of the plurality of sequencing libraries of oligonucleotides, wherein
the sample index oligonucleotides in each of the plurality of sets of sample index oligonucleotides are different from sample index oligonucleotides in each other set of sample index oligonucleotides; and
each sample index oligonucleotide sequence within a set of sample index oligonucleotides comprises a different nucleotide sequence from each other sample index oligonucleotide in the set of sample index oligonucleotides.

12. The method of claim 11, wherein each sample index oligonucleotide sequence within a set of sample index oligonucleotide sequences comprises a different nucleotide at each sequence position from each other sample index oligonucleotide in the set of sample index oligonucleotides.

13. The method of claim 11, wherein the sets of sample index oligonucleotides comprise at least about 10 sets, 20 sets, 50 sets, or 100 sets.

14. The method of claim 11, wherein each set of sample index oligonucleotides has complete diversity from the other sets of sample index oligonucleotides.

15. The method of claim 11, wherein each sample index oligonucleotide within a set of sample index oligonucleotides does not share a common 4-mer sequence with any other sample index oligonucleotide within that same set of sample index oligonucleotides.

16. The method of claim 11, wherein sample index oligonucleotides within a set have less than 80% common bases at common sequence positions with other sample index oligonucleotides within the same set.

17. The method of claim 11, wherein the sample index oligonucleotides are from about 4 to about 10 bases in length.

18. The method of claim 17, wherein sample index oligonucleotides of different sets have different lengths.

19. The method of claim 11, wherein subsequent to the attaching step, the sequencing libraries of oligonucleotides are pooled together and subjected to a sequencing process.

20. The method of claim 11, wherein the sample index oligonucleotide sequences are further integrated into adapter sequences comprising additional sequence elements.
